(19) United States
(12) Patent Application Publication
(10) Pub. No.: US 2006/0263394 A1
(43) Pub. Date: Nov. 23, 2006

(54) Environmental raw material capable of providing impact resistance, method of producing the same, and moldings

(75) Inventors: Hideko Oyama, Tokyo (JP); Yuuko Oono, Tsukuba-shi (JP)

Correspondence Address: BIRCH STEWART KOLASCH & BIRCH, PO BOX 747, FALLS CHURCH, VA, 22040-0747, US

(73) Assignee: National Institute of Advanced Industrial Science and Technology, Tokyo (JP)

(21) Appl. No.: 11/392,725

(22) Filed: Mar. 30, 2006

(30) Foreign Application Priority Data
May 23, 2005 (JP) 2005-150023

(51) Int. Cl. B32B 5/02; C08L 51/00; A61K 9/00

(52) U.S. Cl. 424/400; 442/120; 428/34.1; 525/80

(57) ABSTRACT

A resin composition containing a crystal structure of poly(lactic acid), which is obtained by melt-kneading 30 to 99 mass % of poly(lactic acid) and 70 to 1 mass % of a copolymer having a functional group reactive with the poly(lactic acid); a method of producing the same; and moldings.

### FIELD OF THE INVENTION

The present invention relates to an environmental raw material capable of providing impact resistance, a method of producing the same, and moldings.

### BACKGROUND OF THE INVENTION

Recently, development in biodegradable plastics has advanced actively.

Of the biodegradable plastics, a poly(lactic acid) resin (also referred to as PLA) has advantages such as excellent transparency, rigidity, and workability compared with those of other biodegradable plastics. However, brittleness of the poly(lactic acid) resin is noted as a problem, and the poly(lactic acid) resin is known as a resin formed into a film lacking mechanical strength.

Thus, improvements have been made to produce a resin having high nerve by preparing a copolymer of the poly(lactic acid) and another polyester compound or the like and subjecting the copolymer to a stretching processing. Then, the resultant is biaxially stretched to improve mechanical strength, to thereby obtain physical properties allowing use as a film. It is known that such a film is a heat-shrinkable film and can give dimensional stability through heat treatment (e.g., JP-A-6-23836 (“JP-A” means unexamined published Japanese patent application), JP-A-7-207041, and JP-A-7-256753). The films disclosed in the patent publications are each a biaxially stretched film produced through a tenter method, and are improved in tensile break strength and elongation at break. However, only films with poor tear strength are provided. Further, there are reported: an easily tearable biaxially stretched film comprising a poly(lactic acid)-based polymer and crystalline aliphatic polyester (e.g., JP-A-2000-198913); and an easily tearable poly(lactic acid)-based biaxially stretched film comprising poly(lactic acid) with polyethylene terephthalate and/or polyethylene isophthalate (e.g., JP-A-2001-64413). These films are excellent in linearity in tearing and hand tearing property. However, they are poor in tear strength.

Further, there are reported: a stretched film having excellent dimensional stability, high-speed cutting property and impact resistance, and comprising poly(lactic acid) and aliphatic polyester (glass transition temperature Tg of 10° C. or lower) obtained through polycondensation of aliphatic dicarboxlyic acid and aliphatic diol, or the like (e.g., JP-A-2003-286354); and a film having high tear strength measured in accordance with JIS K 7128 and high impact strength measured in accordance with ASTM-D1709-91 (JP-A-2003-292642).

Further, there is reported a biaxially stretched film containing a poly(lactic acid)-based polymer and aliphatic-aromatic copolymerized polyester, and having a value within a specific difference between heat-shrinkage in a longitudinal direction and heat-shrinkage in a cross direction, which is produced by drawing at 100° C. or lower (e.g., JP-A-2003-342391).

Further, there is reported a film improved in impact resistance by mixing a segmented polyester, a natural rubber or a styrene-butadiene copolymer with poly(lactic acid) (e.g., Japanese Patent No. 2725870). However, these materials and the poly(lactic acid) generally have poor compatibility with each other. Therefore, although impact resistance is improved, a non-uniform blend is liable to be produced in the film. As a result, the film has not only poor appearance but also unstable mechanical strength.

There is reported a method involving: adding epoxidized isoprene, which is an additive for improving molding property, to melted poly(lactic acid); and kneading the mixture (e.g., JP-A-2000-95898).

In a method of heating natural rubber and synthetic rubber for plasticization, adding powder of poly(lactic acid), kneading them at a temperature lower than a melting point of the poly(lactic acid) to form a mixture, and kneading the mixture at a temperature equal to or higher than the melting point of the poly(lactic acid), complex operations are required (e.g., JP-A-2004-143315).

Improvements in melting property, mechanical property, impact resistance, appearance and the like of moldings have been attempted by melt-mixing a polymer-blended material containing poly(lactic acid), by melt-mixing poly(lactic acid) and polyurethane, or by melt-mixing poly(lactic acid) and an epoxy group-containing thermoplastic elastomer (e.g., JP-A-2002-37995). However, impact strength and the like have improved at most twice. Further, there are reported: a composition of poly(lactic acid) and an epoxidized diene-based block copolymer; and a composition prepared by further adding polycaprolactone to the above composition of poly(lactic acid) and an epoxidized diene-based block copolymer (e.g., JP-A-2000-219803). Studies in mechanical strength and biodegradability of molding comprising any one of the compositions have been conducted. JP-A-2000-219803 reports that an amount of the diene-based block copolymer must be increased to improve impact resistance, but the increased amount causes degradation in biodegradability.

A polymer composition comprising of poly(lactic acid) and an epoxy group-containing resin has a uniform composition. However, moldings comprising the composition have insufficient properties such as tensile strength, elongation at break, and impact strength for practical use. In view of such background, there is desired a material having improved physical properties such as tensile strength, elongation at break, and impact strength.

Regarding a polymer composition and resin containing poly(lactic acid), which is a biodegradable material, preparation of a material containing essentially brittle poly(lactic acid), having properties such as excellent tensile strength, elongation at break and flexibility, and being non-broken in an impact resistance test, has seemed impossible. An invention challenging the above-mentioned point has been desired.

### SUMMARY OF THE INVENTION

The present invention resides in a resin composition containing a crystal structure of poly(lactic acid), which is obtained by melt-kneading 30 to 99 mass % of poly(lactic acid) (A) and 70 to 1 mass % of a copolymer (B) having a functional group reactive with the poly(lactic acid) (A).

Further, the present invention resides in a method of producing a resin composition by melt-kneading 30 to 99 mass % of poly(lactic acid) (A) and 70 to 1 mass % of a copolymer (B) having a functional group reactive with the poly(lactic acid), and molding the kneaded composition under conditions for crystal growth.

Further, the present invention resides in moldings obtained from the resin composition described above.

Other and further features and advantages of the invention will appear more fully from the following description, taken in connection with the accompanying drawings.

### BRIEF DESCRIPTION OF THE DRAWINGS

FIG. 1a is an electron microscope photograph of a resin composition used in Example 1.

FIG. 1b is an electron microscope photograph of a resin composition used in Example 5.

FIG. 1c is an electron microscope photograph of a resin composition used in Example 6.

FIG. 2 is wide angle X-ray diffraction (WAXD) spectra (transmission method), in which (a) is a WAXD spectrum of PLA-1 used in Comparative example 1, (b) is a WAXD spectrum of PLA-2 used in Comparative example 2, (c) is a WAXD spectrum of a poly(lactic acid)-containing resin composition formed of PLA-1/Copolymer-1 (weight ratio of 80/20), which was used in Example 1, and (d) is a WAXD spectrum of a poly(lactic acid)-containing resin composition formed of PLA-2/Copolymer-1 (weight ratio of 80/20), which was used in Example 2.

### DETAILED DESCRIPTION OF THE INVENTION

According to the present invention, there is provided the following means:

(1) A resin composition containing a crystal structure of poly(lactic acid), which is obtained by melt-kneading 30 to 99 mass % of poly(lactic acid) (A) and 70 to 1 mass % of a copolymer (B) having a functional group reactive with the poly(lactic acid) (A).

(2) The resin composition according to the above item (1), wherein the copolymer (B) is discontinuously dispersed as a dispersed phase with an average particle size of 10 μm to 10 nm in a continuous phase comprising the poly(lactic acid) (A).

(3) The resin composition according to the above item (1) or (2), wherein an X-ray diffraction profile of the resin composition designates a diffraction pattern with a peak that is characteristic of a crystalline substance of poly(lactic acid), when an X-ray diffraction strength of the resin composition is measured based on an angle (2θ) of a counter tube.

(4) The resin composition according to the above item (1) or (2), wherein a Debye ring is observed when the resin composition is irradiated with X-ray and the diffracted X-ray is received by a flat film.

(5) The resin composition according to the above item (1) or (2), wherein a heat of crystal fusion of the poly(lactic acid) (A) is 15 J/g or more in a DSC measurement at a scan rate of 10° C./minute.

(6) The resin composition according to any one of the above items (1) to (5), wherein the poly(lactic acid) (A) has a weight average molecular weight of 5,000 to 1,000,000.

(7) The resin composition according to any one of the above items (1) to (6), wherein the copolymer (B) is a copolymer having an epoxy group.

(8) The resin composition according to any one of the above items (1) to (7), wherein the copolymer (B) comprises 0.1 to 30 mass % of an unsaturated glycidyl carboxylate unit and/or unsaturated glycidyl ether unit.

(9) The resin composition according to any one of the above items (1) to (8), wherein the copolymer (B) has a structure of an olefin-based compound.

(10) The resin composition according to any one of the above items (1) to (9), wherein the copolymer (B) is an epoxy group-containing ethylene copolymer comprising 60 to 99 mass % of an ethylene unit (a), 0.1 to 20 mass % of an unsaturated glycidyl carboxylate unit and/or unsaturated glycidyl ether unit (b), and 0 to 40 mass % of an ethylene-series unsaturated ester compound (c).

(11) The resin composition according to any one of the above items (1) to (10), wherein a heat of fusion of the copolymer (B) is less than 3 J/g.

(12) The resin composition according to any one of the above items (1) to (11), wherein a Mooney viscosity of the copolymer (B) is 3 to 70.

(13) The resin composition according to any one of the above items (1) to (12), wherein the copolymer (B) is rubber.

(14) The resin composition according to the above item (13), wherein the rubber comprises of a (meth)acrylate-ethylene-(unsaturated glycidyl carboxylate and/or unsaturated glycidyl ether) copolymer.

(15) The resin composition according to the above item (14), wherein the (meth)acrylate contains at least one selected from the group consisting of methyl acrylate, methyl methacrylate, n-butyl acrylate, n-butyl methacrylate, tert-butyl acrylate, tert-butyl methacrylate, 2-ethylhexyl acrylate, and 2-ethylhexyl methacrylate.

(16) The resin composition according to any one of the above items (1) to (15), wherein the copolymer (B) is a block copolymer having a functional group reactive with poly(lactic acid) and comprising a block having at least one structure of a vinyl aromatic compound and a block having at least one structure of a conjugated diene compound.

(17) The resin composition according to any one of the above items (1) to (16), wherein the copolymer (B) is a styrene-based elastomer.

(18) The resin composition according to any one of the above items (1) to (17), wherein the copolymer (B) is a triblock copolymer having an epoxy group.

(19) A method of producing a resin composition, comprising the steps of:

melt-kneading 30 to 99 mass % of poly(lactic acid) (A) and 70 to 1 mass % of a copolymer (B) having a functional group reactive with the poly(lactic acid); and

molding the kneaded composition under conditions for crystal growth.

(20) The method of producing a resin composition according to the above item (19), wherein a molding temperature is adjusted within a range of ±30° C. of a peak crystallization temperature of the resin composition, in which the peak crystallization temperature is obtained by reducing a temperature in a molten state of the resin composition through a differential scanning calorimetry (DSC).

(21) The method of producing a resin composition according to the above item (19) or (20), wherein the obtained resin composition is subjected to a heat treatment after molding, to accelerate crystallization.

(22) Moldings, which are obtained by molding the resin composition according to any one of the above items (1) to (18) or the resin composition obtained by the producing method according to any one of the above items (19) to (21).

(23) The moldings according to the above item (22), wherein the moldings are obtained by adjusting a molding temperature within a range of ±30° C. of a peak crystallization temperature of the resin composition, in which the peak crystallization temperature is obtained by reducing a temperature in a molten state of the resin composition through a differential scanning calorimetry (DSC).

(24) The moldings according to the above item (22) or (23), wherein the obtained moldings are subjected to a heat treatment after molding.

(25) The moldings according to any one of the above items (22) to (24), wherein the molding is injection molding.

(26) The moldings according to any one of the above items (22) to (24), wherein the molding is press molding.

(27) The moldings according to any one of the above items (22) to (24), wherein the molding is extrusion molding.

(28) The moldings according to any one of the above items (22) to (24), wherein the molding is film molding.

(29) The moldings according to any one of the above items (22) to (28), wherein a shape of the moldings is any one of a film, a sheet, and a plate.

(30) The moldings according to any one of the above items (22) to (28), wherein a shape of the moldings is any one of a net, a fiber, a non woven fabric, a woven fabric, and a filament.

(31) The moldings according to any one of the above items (22) to (28), wherein a shape of the moldings is a rod or an irregular shape.

(32) The moldings according to any one of the above items (22) to (28), wherein a shape of the moldings is any one of a tube, a pipe, a bottle, and a cylinder.

(33) The moldings according to any one of the above items (22) to (32), wherein the moldings are used for any one of containers, home appliance components, transmission components, housing materials, toys, commodities, materials for agriculture or fishery, mobile device components, packaging materials, medical components, and automobile components.

The inventors of the present invention have found that a mixture containing 30 to 99 mass % of poly(lactic acid) (A) and 70 to 1 mass % of a copolymer (B) having a functional group reactive with the poly(lactic acid) is melt-kneaded and molded under conditions for crystallization, to thereby provide a resin composition having significantly improved ductility, toughness, flexibility, and impact resistance. The inventors of the preset invention have confirmed that the resin composition of the present invention is improved in impact resistance without degradation of heat stability, compared with that of a resin composition formed solely of poly(lactic acid). As a result, the inventors have completed the present invention.

Herein, the resin composition and the resin composition given by the producing method of the present invention in a certain shape can be used for a desired use as they are. Further, they can be also used as a raw material to form moldings.

For production of the resin composition of the present invention, a mixing ratio A/B of poly(lactic acid) (A) to a copolymer (B) having a functional group reactive with the poly(lactic acid) is adjusted to 99.0/1.0 to 30.0/70.0 (mass ratio), and preferably 95/5 to 50/50 (mass ratio). On terminals of a chain of the poly(lactic acid) (A), a hydroxyl group and/or a carboxylic acid group are present. Thus, in the presence of another functional group, in the copolymer (B), causing a coupling reaction, an addition reaction, or the like with the hydroxyl group and/or the carboxylic acid group at a high temperature for melt-kneading, a copolymer (A-B) containing the poly(lactic acid) (A) and the copolymer (B) is newly formed at an interface. The copolymer (A-B) serves as an emulsifier for the incompatible resin composition, to thereby contribute in dispersion stabilization, improvement of interface adhesiveness, improvement of mechanical properties, and the like. Then, the obtained resin mixture is molded under conditions for crystal growth (conditions and methods are not limited), to thereby provide the resin composition of the present invention.

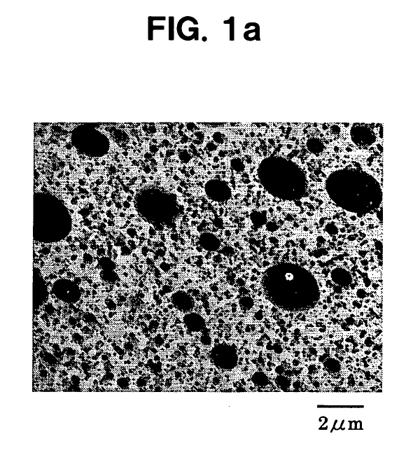

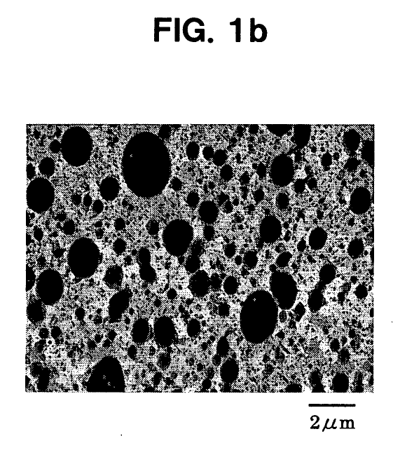

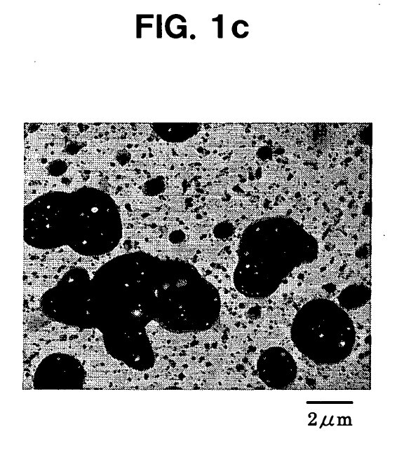

The resin composition obtained through melt-kneading preferably includes the copolymer (B) discontinuously dispersed as a dispersed phase of an average particle size of 10 μm to 10 nm, more preferably 2 μm to 100 nm, in a continuous phase comprising the poly(lactic acid) (A). The average particle size of the reactive copolymer (B) varies depending on the molecular weight and melt viscosity of the component polymers, the concentration, kind, and position in a polymer chain of the reactive group in the reactive copolymer (B), the molding conditions, and the like. Too large a particle size degrades mixing properties of both component polymers, and properties provided in the present invention are not developed. In consideration of dimensions of the polymer chain, a dispersed phase of an average particle size of 10 nm or less is hardly obtained by melt-kneading. In this connection, see EXAMPLES and FIG. 1a to FIG. 1c of the present specification.

For formation of the continuous phase of poly(lactic acid), a weight of the poly(lactic acid) not necessarily needs to be 50% or more of the total weight of the component polymers. The reactive copolymer having a higher melt viscosity than that of the poly(lactic acid) at a kneading temperature may provide a continuous phase of the poly(lactic acid) even if the weight of poly(lactic acid) is 50% or less. Biodegradability degrades significantly with a discontinuous phase of the poly(lactic acid).

When an X-ray diffraction profile of the resin composition of the present invention designates a diffraction pattern with a peak that is characteristic of a crystalline substance of poly(lactic acid), it can be confirmed that the resin composition is a crystalline resin composition. Specifically, when an X-ray diffraction strength of the resin composition is measured based on an angle (2θ) of a counter tube, a diffraction pattern having a sharp peak derived from the crystalline substance of poly(lactic acid) appears.

In addition, when the resin composition of the present invention is irradiated with X-ray and the diffracted X-ray is received by a flat film, a Debye ring is preferably observed, which is one of characteristics of a crystalline substance. Thus, the crystalline substance of poly(lactic acid) can also be confirmed.

Further, the presence of the crystalline substance of poly(lactic acid) can also be confirmed by a differential scanning calorimetry (DSC). When a DSC measurement is carried out at a scan rate of 10° C./minute, a heat of crystal fusion of the poly(lactic acid) (A) is preferably 15 J/g or more, more preferably 30 J/g or more. When the amount of produced crystals is large, the resin composition is improved in heat resistance and impact resistance. In particular, the heat of crystal fusion is further preferably 35 J/g or more. In this case, when crystals are formed at the time of increasing the temperature in the DSC measurement, the term “heat of crystal fusion” means a heat of fusion of the crystalline substance calculated by excluding the effect of the formation of the crystals.

Poly(lactic acid) has three kinds of crystal structures of α-type structure, β-type structure, and γ-type structure. The α-type structure is a most stable structure. The β-type structure develops when poly(lactic acid) is intensively stretched at a high temperature. The γ-structure develops in epitaxial crystallization.

Formation of a crystal structure in a resin composition can be confirmed through heat analysis or the like. However, the formation of a crystal structure can be confirmed easily through X-ray diffraction measurement of the resin composition, because a sharp specific peak (crystal structure not specified) derived from a crystalline substance of poly(lactic acid) appears in a diffraction pattern. Upon formation of a crystal structure, ductile property degrades drastically and impact resistance improves significantly, compared with the case of an amorphous resin (comparison with Japanese Patent Application No.2004-342958).

Description of poly(lactic acid) (A) will be given below.

Lactic acid has optical isomers of L-lactic acid and D-lactic acid. Examples of poly(lactic acid) produced through polymerization of those optical isomers include: crystalline poly(lactic acid) containing about 10% or less of a D-lactic acid unit and about 90% or more of an L-lactic acid unit or containing about 10% or less of an L-lactic acid unit and about 90% or more of a D-lactic acid unit, and having an optical purity of about 80% or more; and amorphous poly(lactic acid) containing 10% to 90% of a D-lactic acid unit and 90% to 10% of an L-lactic acid unit, and having an optical purity of about 80% or less. Crystalline poly(lactic acid) (D-type and L-type) having high optical purity is preferably used as a raw material in the present invention for accelerating crystallization.

The poly(lactic acid) preferably has a weight average molecular weight of 5,000 to 1,000,000. Too large a weight average molecular weight and too small a weight average molecular weight provide insufficient effects on a resin composition to be produced. In particular, excessively large a weight average molecular weight provides too low a concentration of the terminal groups of the poly(lactic acid) causing a reaction and too high a melt viscosity, and an interface reaction with the reactive copolymer is hardly caused. The range of the weight average molecular weight is more preferably 20,000 to 700,000, and furthermore preferably 20,000 to 500,000.

A molecular weight of the poly(lactic acid) may be adjusted by adding a small amount of, for example, polyol, glycol or acid, and forming a hindered terminal group of the poly(lactic acid), for improving molding property of the poly(lactic acid). For example, 0.5 to 10 mass % of polyethylene glycol may be copolymerized with the poly(lactic acid) for stable molding.

The poly(lactic acid) (A) that can be used in the present invention preferably has a modulus of flexural rigidity of 8,000 to 45,000 kg/cm² (0.8 to 4.5 GPa). Further, the poly(lactic acid) (A) that can be used in the present invention preferably has a melt index (hereinafter which may be referred to as MFR (melt flow rate), in accordance with JIS K 6760, 190° C., 2.16 kgf) of 1 to 50 g/10 minutes. However, the present invention is not limited to these.

The functional group reactive with the poly(lactic acid) in the copolymer (B) having a functional group reactive with the poly(lactic acid) may be any material as long as it has reactivity with the terminal groups (hydroxyl group or carboxyl group) of the poly(lactic acid) (A). Examples thereof include an epoxy group, an amino group, an isocyanate group, an oxazoline group, a carbodiimide group, and an ortho-ester group. The functional group is preferably an epoxy group.

The epoxy group or the like may exist as a part of other functional groups, and an example thereof is a glycidyl group.

As a monomer having such a functional group, a monomer having a glycidyl group is particularly preferably used. Examples of the monomer having a glycidyl group include unsaturated glycidyl ether and unsaturated glycidyl carboxylate represented by the following formula:

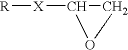

wherein, R represents a hydrocarbon group having 2 to 13 carbon atoms and having an ethylene-series unsaturated bond; and X represents —C(O)O—, —CH₂—O—, or the following group.

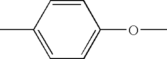

Examples of the unsaturated glycidyl carboxylate include glycidyl acrylate, glycidyl methacrylate, diglycidyl itaconate, butene triglycidyl tricarboxylate, and p-styrene glycidyl carboxylate.

Examples of the unsaturated glycidyl ether include vinyl glycidyl ether, allyl glycidyl ether, 2-methyl allyl glycidyl ether, methacryl glycidyl ether, and styrene-p-glycidyl ether.

In the present invention, the copolymer (B) contains preferably 0.1 to 30 mass %, and more preferably 0.1 to 20 mass % of the unsaturated glycidyl carboxylate unit and/or unsaturated glycidyl ether unit.

A method of introducing such a functional group into the copolymer (B) is not particularly limited, and a known method can be employed. For example, the functional group may be introduced into the copolymer through copolymerization of a monomer having the functional group in the step of synthesis of the copolymer. Further, the functional group may be introduced into the copolymer through graft copolymerization of a monomer having the functional group. Alternatively, the functional group, such as an epoxy group, may be introduced into the copolymer through a chemical reaction of an unsaturated bond.

The copolymer (B) having a functional group reactive with the poly(lactic acid) (A) may be a thermoplastic resin, rubber, or a mixture of a thermoplastic resin and rubber.

When the copolymer (B) having a functional group reactive with the poly(lactic acid) (A) is a resin or contains a resin, it may contain an olefin-based compound.

Examples of the copolymer (B) as the thermoplastic resin include a resin comprising a copolymer containing an ethylene unit (a), an unsaturated glycidyl carboxylate unit and/or unsaturated glycidyl ether unit (b), and an ethylene-series unsaturated ester compound (c). In particular, an epoxy group-containing ethylene copolymer containing 60 to 99 mass % of the ethylene unit (a), 0.1 to 20 mass % of the unsaturated glycidyl carboxylate unit and/or unsaturated glycidyl ether unit (b), and 0 to 40 mass % of the ethylene-series unsaturated ester compound (c) is preferable. Further, an epoxy group-containing ethylene copolymer containing 60 to 98 mass % of the ethylene unit (a), 0.1 to 15 mass % of the unsaturated glycidyl carboxylate unit and/or unsaturated glycidyl ether unit (b), and 0 to 35 mass % of the ethylene-series unsaturated ester compound (c) is more preferable.

The copolymer that can be used in the present invention may comprise a component containing an acetyl group, such as vinyl acetate, in a small amount (e.g., 0 to 10 mass %).

Definitions of the unsaturated glycidyl carboxylate unit and the unsaturated glycidyl ether unit and specific examples thereof are the same as those described above.

Examples of the ethylene-series unsaturated ester compound (c) include vinyl carboxylates such as vinyl acetate, vinyl propionate, methyl acrylate, ethyl acrylate, butyl acrylate, methyl methacrylate, ethyl methacrylate, and butyl methacrylate, and an alkyl α,β-unsaturated carboxylate. Vinyl acetate, methyl acrylate, and ethyl acrylate are particularly preferable.

Examples of the epoxy group-containing ethylene copolymer include a copolymer comprising an ethylene unit and a glycidyl methacrylate unit; a copolymer comprising an ethylene unit, a glycidyl methacrylate unit, and a methyl acrylate unit; and a copolymer comprising an ethylene unit, a glycidyl methacrylate unit, and a vinyl acetate unit.

The epoxy group-containing ethylene copolymer preferably has a modulus of flexural rigidity of 10 to 1,300 kg/cm², and more preferably 20 to 1,100 kg/cm². Too large a modulus of flexural rigidity and too small a modulus of flexural rigidity may provide insufficient mechanical properties of moldings prepared by using the resin composition on occasion.

The copolymer (B) is preferably a copolymer having a heat of crystal fusion of less than 3 J/g, for providing the moldings of the present invention with good heat stability and flexibility. When the heat of crystal fusion is too large due to crystallization advanced in excess, the copolymer (B) cannot play a role as an external-energy-absorbing agent.

The epoxy group-containing ethylene copolymer can be produced through a method generally involving: mixing of a high pressure radical-generating agent for copolymerization of an unsaturated epoxy compound and ethylene at 500 to 4,000 atmospheric pressure, at 100 to 300° C., and in the presence or absence of an appropriate solvent or chain transfer agent; and conducting a melt graft copolymerization of the unsaturated epoxy compound and ethylene in an extruder.

A melt index (hereinafter which may be referred to as MI or MFR (melt flow rate), in accordance with JIS K 6760, 190° C., 2.16 kgf) of the epoxy group-containing ethylene copolymer is preferably 0.5 to 100 g/10 minutes, and more preferably 2 to 50 g/10 minutes. Too large melt index is not preferred from the viewpoint of mechanical properties. Too small melt index is not preferred because compatibility of the epoxy group-containing ethylene copolymer with the poly(lactic acid) (A) is poor.

The copolymer (B) has a Mooney viscosity of preferably 3 to 70, more preferably 3 to 30, and particularly preferably 4 to 25. The Mooney viscosity as used herein refers to a value measured in accordance with JIS K 6300 at 100° C. by using a large rotor.

In the case where the copolymer (B) is rubber or contains rubber, a method of introducing the functional group into the rubber in the composition is not particularly limited, and a known method can be employed. For example, a monomer having the functional group may be introduced into the rubber through copolymerization in synthesis of the rubber. Alternatively, a monomer having the functional group may be introduced into the rubber through graft copolymerization.

Specific examples of the rubber include (1) (acryl)rubber having an epoxy group, and (2) a block copolymer having a functional group reactive with poly(lactic acid) and comprising a block having at least one structure of a vinyl aromatic hydrocarbon compound and a block having at least one structure of a conjugated diene compound (hereinafter, a block copolymer comprising a block having at least one structure of a vinyl aromatic hydrocarbon compound and a block having at least one structure of a conjugated diene compound is also referred to as “(vinyl aromatic hydrocarbon compound)-(conjugated diene compound) block copolymer”).

An example of the (acryl)rubber having an epoxy group in the above-described specific example (1) of the rubber includes a (meth)acrylate (or methacrylate)-ethylene-(unsaturated glycidyl carboxylate and/or unsaturated glycidyl ether) copolymer rubber.

Examples of the (meth)acrylate include methyl acrylate, methyl methacrylate, n-butyl acrylate, n-butyl methacrylate, tert-butyl acrylate, tert-butyl methacrylate, 2-ethylhexyl acrylate, and 2-ethylhexyl methacrylate.

Examples of the (vinyl aromatic hydrocarbon compound)-(conjugated diene compound) block copolymer having a functional group reactive with poly(lactic acid) as mentioned in the above specific example (2) of the rubber include as below.

Examples of the vinyl aromatic hydrocarbon compound in the (vinyl aromatic hydrocarbon compound)-(conjugated diene compound) block copolymer rubber include styrene, vinyl toluene, divinyl benzene, α-methyl styrene, p-methyl styrene, and vinyl naphthalene. Of these, styrene is preferable. Example of the conjugated diene compound include butadiene, isoprene, 1,3-pentadiene, and 3-butyl-1,3-octadiene; and butadiene and isoprene are preferable.

The (vinyl aromatic hydrocarbon compound)-(conjugated diene compound) block copolymer, or a hydrogenated product thereof can be prepared through known methods such as those described, for example, in JP-B-40-23798 (“JP-B” means examined Japanese patent publication) and JP-A-59-133203.

The (vinyl aromatic hydrocarbon compound)-(conjugated diene compound) block copolymer rubber having a functional group reactive with poly(lactic acid) can be obtained by introducing a monomer having a functional group reactive with poly(lactic acid), such as an epoxy group, into the (vinyl aromatic hydrocarbon compound)-(conjugated diene compound) block copolymer obtained through the methods described above. A method of introducing such a monomer into the (vinyl aromatic hydrocarbon compound)-(conjugated diene compound) block copolymer is not particularly limited, but graft copolymerization is preferably employed.

The copolymer (B) may be a styrene-based elastomer. An example of the styrene-based elastomer is a styrene-butadiene block copolymer. In the styrene-butadiene block copolymer, the functional group reactive with poly(lactic acid) such as an epoxy group may exist in the styrene block or the butadiene block. In the present invention, the epoxy group preferably exists on a side chain than on a main chain of butadiene.

A styrene content in the styrene-butadiene block copolymer is preferably 5 to 80 mass %, and more preferably 2 to 50 mass %.

The copolymer (B) having a functional group reactive with poly(lactic acid) may be a triblock copolymer having a functional group, such as an epoxy group, reactive with poly(lactic acid).

An example of the triblock copolymer as the component (B) defined in the present invention is a styrene-ethylene/butylene-styrene block copolymer having a functional group reactive with poly(lactic acid).

The rubber used as the copolymer (B) defined in the present invention may be vulcanized as required, and be used as vulcanized rubber.

Vulcanization of acrylate (or methacrylate)-ethylene-(unsaturated glycidyl carboxylate and/or unsaturated glycidyl ether) copolymer rubber is performed by using a polyfunctional organic acid, a polyfunctional amine compound, an imidazole compound, or the like, but a vulcanization method is not limited thereto.

A method of producing a resin composition of the present invention involves kneading (melt-kneading) of each component of a pelletized mixture in a molten state containing the poly(lactic acid) (A) and the copolymer (B). For melt-kneading, generally used kneaders such as a single-screw or twin-screw extruder and various kneaders can be used. Of those, the twin-screw extruder is preferred. A melt-kneading temperature is generally about 180 to 250° C.

A resin composition containing the copolymer (B) having a functional group reactive with the poly(lactic acid) (A) finely dispersed in a matrix of the poly(lactic acid) (A) can be obtained through melt-kneading.

In melt-kneading, each component may be mixed uniformly by using an apparatus such as a tumblering mixer or a Henschel mixer in advance, and then supplied to a kneader. Alternatively, a method of separately and quantitatively supplying each component into a kneader may also be employed.

A temperature inside the tumbling mixer or the Henschel mixer is generally 180° C. or higher. The temperature may generally be about 210° C., and a high temperature of 280° C. or higher must be avoided.

The resin composition containing the poly(lactic acid) (A) and the copolymer (B) having a functional group reactive with the poly(lactic acid) (A) may contain, as well as a lubricant, a crystallization accelerator as a nucleating agent on crystallization, as required.

A mixing amount of the crystallization accelerator that may be used in the present invention is preferably 30 to 0.01 parts by weight, more preferably 20 to 0.05 parts by weight, and furthermore preferably 10 to 0.05 parts by weight with respect to 100 parts by weight of the resin composition containing the poly(lactic acid) (A) and the copolymer (B) having a functional group reactive with the poly(lactic acid) (A).

A material generally used as a nucleating agent on crystallization for a polymer can be used as the nucleating agent on crystallization as the crystallization accelerator that can be used in the present invention without particular limitation. Both an inorganic nucleating agent on crystallization and an organic nucleating agent on crystallization may be used.

Specific examples of the inorganic nucleating agent on crystallization include talk, kaolin, montmorillonite, synthetic mica, clay, zeolite, silica, graphite, carbon black, zinc oxide, magnesium oxide, titanium oxide, calcium sulfate, boron nitride, calcium carbonate, barium sulfate, aluminum oxide, neodymium oxide, and a metal salt of phenyl phosphate. These inorganic crystal nucleating agents are preferably modified with an organic substance for enhancing their dispersibility in the resin composition.

Specific examples of the organic nucleating agent on crystallization include organic metal carboxylates such as sodium benzoate, potassium benzoate, lithium benzoate, calcium benzoate, magnesium benzoate, barium benzoate, lithium terephthalate, sodium terephthalate, potassium terephthalate, calcium oxalate, sodium laurate, potassium laurate, sodium myristate, calcium myristate, sodium octacosanoate, calcium octacosanoate, sodium stearate, potassium stearate, lithium stearate, calcium stearate, magnesium stearate, barium stearate, sodium montanate, calcium montanate, sodium toluinate, potassium salicylate, sodium salicylate, zinc salicylate, aluminum dibenzoate, potassium dibenzoate, lithium dibenzoate, sodium β-naphthalate, and sodium cyclohexane carboxylate; organic sulfonates such as sodium p-toluene sulfonate and sodium sulfoisophthalate; carboxylic amides such as stearamide, ethylene bislauramide, paltimic acid amide, hydroxy stearamide, erucamide, and trimesic tris (t-butylamide); polymers such as low density polyethylene, high density polyethylene, polypropylene, polyisopropylene, polybutene, poly-4-methyl pentene, poly-3-methyl butene-1, polyvinylcycloalkane, polyvinyltrialkylsilane, and poly(lactic acid) having a high-melting point; a sodium salt of a polymer having a carboxyl group such as a sodium salt of an ethylene-acrylate or ethylene-methacrylate copolymer and a sodium salt of a styrene-maleic anhydride copolymer, or a potassium salt thereof (so called ionomer); benzylidene sorbitol and a derivative thereof; a phosphorus compound metal salt such as methylenebis(2,4-di-t-butylphenyl)phosphate; and 2,2-methylbis(4,6-di-t-butylphenyl)sodium.

In the present invention, the resin composition can be preferably obtained via any of the following two kinds of crystallizing methods. One crystallizing method includes: adjusting a molding temperature within a range of ±30° C. of a peak crystallization temperature of the resin composition, in which the peak crystallization temperature is obtained by reducing a temperature in a molten state of the resin composition, at a rate of 10 to 20° C./minute through a differential scanning calorimetry (DSC), to thereby crystallize and mold the resin composition. In the crystallizing step, injection molding is a preferred molding method.

Another crystallizing method includes: subjecting solidified moldings to heat treatment, to accelerate crystallization of the poly(lactic acid), which method is preferably employed for improving the heat resistance and impact resistance of the moldings. To be specific, the solidified moldings are heated to a temperature between a glass transition temperature (about 60° C.) and a melting point (about 170° C.), preferably a temperature between a start point and a finish point of the crystallization peak, of the resin composition measured by using a differential scanning calorimeter (DSC). Preferably, the moldings are heated within a range of ±30° C. of a peak crystallization temperature of the resin composition, in which the peak crystallization temperature is obtained by reducing a temperature in a molten state through a differential scanning calorimetry (DSC).

Examples of a method of accelerating crystallization include, in the case that the resin composition in a molten state is cooled down to crystallize the resin composition, a method in which a mold is kept for a certain period of time at a temperature for crystallizing the resin composition, and the resin composition is crystallized in the mold; and, in the case that the solidified moldings are subjected to the heat treatment, a method in which the heat treatment is carried out in an oven. However, the present invention is not limited to these methods, as long as these methods and treatment time are sufficient to cause crystallization of the poly(lactic acid).

The above-described resin composition may be molded into a desired shape through various known molding methods, to thereby obtain various moldings. Specific examples of the molding method are shown below.

Specific examples thereof include extrusion molding, injection molding, rotational molding, blow molding, transfer molding, press molding, solvent casting, and film molding.

Moldings to be obtained through these molding methods are referred to as extrusion moldings, injection moldings, rotational moldings, blow moldings, transfer moldings, press moldings, solvent cast moldings, and film moldings, corresponding to the respective molding methods.

When a sample obtained from the resin composition of the present invention by injection molding or the like is subjected to an impact test according to JIS K 7111 (ISO 179) or the like, the sample with notch may have a Charpy impact value about 15 to 60 times to the sample obtained solely from poly(lactic acid). This is shown in Table 1 described below. This value is comparable to several times that of an ABS resin which is a standard resin derived from petroleum. This property may be utilized, and the moldings of the present invention may be used as a safe and hardly broken material for a wide range of uses such as automobile components, home appliance components, housing materials, baby products, and toys.

When a sample obtained from the resin composition of the present invention by injection molding or the like is subjected to a test according to JIS K 7113 or the like, it has elongation at break several times that of a resin composition obtained solely from poly(lactic acid). This is shown in Table 1. Thus, it is seen that, according to the present invention, essentially brittle poly(lactic acid) is modified to a material that is excellent in ductility.

When a sample obtained from the resin composition of the present invention by injection molding or the like is subjected to a test according to JIS K 7191-1 (ISO 75-1) or the like, it can be seen that when a heat distortion temperature of a resin composition obtained solely from poly(lactic acid) is represented by “T”, a heat distortion temperature of the sample of the present invention is in a range of from (T−5)° C. to (T+5)° C. This is shown in Table 1. Specifically, the heat distortion temperature of the sample of the present invention was higher by 4° C. to 5° C. than that of the sample obtained solely from poly(lactic acid) which was not subjected to a heat processing, and lower by about 2° C. than that of the sample obtained solely from poly(lactic acid) which was subjected to a heat processing. From the results, it can be seen that the heat stability of the poly(lactic acid)-containing resin composition of the present invention is equal to that of the composition formed solely of poly(lactic acid).

To mold the resin composition of the present invention into home appliance components, transmission components, automobile components (such as a bumper and an instrument) and the like, a method in which a molten resin composition is injected from an extruder into a metal mold at high pressure using a molding apparatus such as an injection molding apparatus, can be used.

The resin composition of the present invention may be molded into films through, for example, a method in which the resin composition is supplied to an extruder provided with a die (head).

A T-die or a die with a cylindrical slit is preferably used as a die used for production of the films. Further, casting or heat pressing may also be employed for the production of the films.

A thickness of the moldings molded into films is not particularly limited, but it is preferably 1 to 1,000 μm for practical use, and more preferably 1 to 500 μm. A film surface may be subjected to a surface treatment as required. Examples of the surface treatment include irradiation of α-ray, β-ray, γ-ray, an electron beam, or the like; corona discharge treatment; plasma treatment; flame treatment; infrared treatment; sputtering treatment; solvent treatment; polishing treatment; application or lamination of a resin formed of polyamide, polyolefin, or the like; deposition a metal such as aluminum oxide; and coating of silicon oxide, titanium oxide, or the like.

These treatments may be performed during molding, or performed on a molded film or sheet. However, they are preferably performed during molding, in particular, before a take-up step.

For facilitating passage of a film during film production, an inorganic lubricant such as silica, alumina, and kaolin is preferably added in a required amount for film formation, to thereby provide a film surface with slip property. Further, for improvement of printing property of a film, an antistatic agent or the like may be included in the film.

The resin composition containing poly(lactic acid) (A) and the copolymer (B) having a functional group reactive with the poly(lactic acid) (A) to be used as a film may contain additives such as a metal compound as a static pinning agent, a flame retardant (such as an organophosphorus-based or boric acid-based flame retardant, and aluminum hydroxide), and a defoaming agent as required, in addition to the lubricant.

Further, the resin composition may contain various additives such as an organic filler, an antioxidant, a heat stabilizer (such as a phenol-based, aromatic amine-based, an organosulfur-based, or organophosphorus-based heat stabilizer), a light stabilizer, an inorganic or organic coloring agent, a rust inhibitor, a crosslinking agent, a foaming agent, a fluorescent agent, a surface smoothing agent, a surface gloss improving agent, and a release-improving agent such as a fluorine resin.

A method of adding those additives is not particularly limited and an optimal method in accordance with use may be employed. Additives having heat stability may be added during kneading of a resin. Additives having no heat stability may be added after kneading through application, incorporation, or surface treatment. In the case where small amounts of the additives are kneaded or the additives are added mainly through surface treatment, mechanical properties of the resin composition of the present invention are determined mainly by bulk property. Thus, properties of the moldings such as ductility, toughness, flexibility, and impact resistance are maintained, and the resin composition can be used in fields making use of those properties.

The film to be obtained from the resin composition of the present invention is improved in toughness, flexibility, and impact resistance, has a significantly improved impact value while heat stability is maintained, is modified into a ductile film from a brittle film, and has excellent bending property, oil resistance, adhesiveness, and heat seal property. Thus, by making use of those properties, the film can preferably be used for a food packaging film or tape, a garbage bag, a laminate film, a wrapping film for an electrical and electronic component or the like, a shrink film of a recording medium, a film for agriculture or fishery, or the like.

The resin composition may be molded into a filament through a method in which the resin composition is melt-extruded into a strand die by using an extruder, and taken up at high speed.

The resin composition may be molded into hollow moldings such as a container or a bottle through, for example, a method (blow molding) in which a pressured fluid of air, water, or the like is blown to the resin composition by using a blow molding apparatus or the like, and allowed to adhere into a metal mold. Alternatively, injection molding may also be employed.

The resin composition may be molded into a pipe or a tube through a method in which the resin composition is melt-extruded from an extruder into a tube die by using a tube-molding apparatus.

Examples of a shape of the moldings described above include a film, a sheet, a plate, a net, a fiber, a nonwoven fabric, a woven fabric, a filament, a rod, an irregular shape, a tube, a pipe, a cylinder such as a bottle and a container, and a box.

In the method of producing a resin composition of the present invention, a mold lubricant may be used in the molding process. The mold lubricant is not particularly limited, and any of these ordinary used in the art may be used in the present invention. The mold lubricant is preferably an oleic amide-series lubricant because of its low price. One kind of the mold lubricant may be used singly, or two or more kinds of the mold lubricants may be used in combination. For example, the mold lubricant may be applied on the surface of a mold before a molding step.

As described above, the resin composition of the present invention can be used for moldings as a safe and environmentally-friendly material having excellent mechanical properties, such as toys, baby products, home groceries including containers, home appliance components, office machines, automobile components, medical instruments, electrical and electronic components, AV equipment, nets for fishery field, filters, clothing, and the like.

According to the present invention, it is possible to provide a novel environmentally-friendly raw material containing poly(lactic acid) and having properties such as ductility, toughness, flexibility, and impact resistance; a method of producing the same; and moldings.

The resin composition obtained in the present invention is a resin composition obtained by melt-kneading 30 to 99 mass % of poly(lactic acid) (A) and 70 to 1 mass % of a copolymer (B) having a functional group reactive with the poly(lactic acid) (A), and molding the kneaded product under the conditions for crystallization. The resin composition has significantly improved ductility, toughness, flexibility, and impact resistance. The resin composition of the present invention has improved impact resistance, without degradation of heat stability, than that of a resin composition formed solely of poly(lactic acid).

The resin composition of the present invention is a novel impact-resistant environmentally-friendly raw material to replace conventionally used plastics derived from petroleum. The moldings of the present invention are expected to be used in a wide range of uses such as commodities including toys and baby products, automobile components, electrical and electronic components, clothing, housing materials, medical components, and mobile device components.

The present invention will be described in more detail based on examples given below, but the invention is not meant to be limited by these.

### EXAMPLES

Physical property tests in examples and comparative examples described below were performed through the following methods.

(1) Tensile Test

The resin composition was subjected to injection molding into a multipurpose test piece (thickness of about 4 mm) of ISO 3167 type A, and the tensile test was performed in accordance with JIS K 7113. As a test machine, UTM-III-10T Tensilon (trade name, manufactured by Orientec Co., Ltd.) was used. Measurement was repeated 3 to 5 times for the same sample.

From the above physical property test, tensile modulus and elongation at break were determined.

(2) Flexural Modulus

A multipurpose test piece of ISO 3167 type A was produced through injection molding, and the test piece was measured for a flexural modulus through a three-point flexural test at a test speed of 2 mm/min in accordance with JIS K 7171 (ISO 178). A test machine of the same model as that of the test machine in the above (1) was used.

(3) Measurement of Heat Distortion Temperature (HDT)

A multipurpose test piece of ISO 3167 type A was produced through injection molding. The test piece (thickness of about 4 mm) was cut out in accordance with JIS K 7191 (ISO 75-2), and a test of temperature of deflection under load was performed. With respect to test conditions, a bending stress on the test piece was 1.80 MPa, and the test piece was placed through a flat wise method. A heat distortion test (HDT) machine 3M-2 (trade name, manufactured by Toyo Seiki Co., Ltd.) was used as a test machine.

(4) Charpy Impact Test of Notched Sample

A multipurpose test piece (thickness of about 4 mm) of ISO 3167 type A was produced through injection molding. The test piece was cut out in accordance with JIS K 7111 (ISO 179) and was provided with a notch of type A. The notched test piece was subjected to a Charpy impact test by using a Charpy impact test machine (manufactured by Toyo Seiki Co., Ltd.).

(5) Wide Angle X-Ray Diffraction (WAXD) Measurement

Wide angle X-ray diffraction (WAXD) measurement was performed through a transmission method by using Ultrax 8000 (trade name, manufactured by Rigaku Corporation) under the conditions of Cu Kα-ray source, 40 kV, and 70 mA.

Raw materials used in examples and comparative examples are described below.

Component (A): Poly(lactic Acid) (PLA)

PLA-1: H-100
(trade name, produced by Mitsui Chemicals, Inc., MFR (190° C., 2.16 kgf) of 8 g/10 min, weight average molecular weight Mw of about 140,000 to 150,000, D-type content of 1 to 2%, glass transition temperature of 62.5° C., melting point of 164° C.)

PLA-2: H-400
(trade name, produced by Mitsui Chemicals, Inc., MFR (190° C., 2.16 kgf) of 3 g/10 min, weight average molecular weight Mw of about 200,000, D-type content of 1 to 2%, glass transition temperature of 63° C., melting point of 166° C.)
   Component (B): Composition Reactive with Poly(lactic Acid)

Copolymer-1: Bondfast 7L
(trade name, produced by Sumitomo Chemicals, Co., Ltd., ethylene/methyl acrylate/glycidyl methacrylate=67/30/3 in weight ratio, MFR (190° C., 2.16 kgf) of 9 g/10 min)

Copolymer-2: EPOFRIEND AT501
(trade name, produced by Daicel Chemical Industries, Ltd., styrene-butadiene-styrene block copolymer, styrene/butadiene=40/60 in weight ratio, MFR (190° C., 2.16 kgf) of 7 g/10 min, hydrogenation rate of 0%)

Copolymer-3: EPOFRIEND HT302
(trade name, produced by Daicel Chemical Industries, Ltd., styrene-butadiene-styrene block copolymer, styrene/butadiene=20/80 in weight ratio, MFR (190° C., 2.16 kgf) of 2 g/10 min, hydrogenation rate of about 50%)

Next, description will be given of a shear kneader.

For preparation of samples of a poly(lactic acid)-containing resin compositions, a twin-screw extruder KZW15-30MG (trade name, manufactured by Technovel Corporation, screw diameter of 15 mm, segment type screw, L/D=30) was used as a kneader.

### Example 1

PLA-1 was dried in vacuum at 80° C. for 8 hours, and Copolymer-1 was dried in vacuum at 60° C. for 4 hours. Then, pellets containing 80 parts by weight of the PLA-1 and 20 parts by weight of the Copolymer-1 were mixed and melt-kneaded by using the twin-screw extruder as follows:

The twin-screw extruder equipped with a pressure-reducing vent was heated to 170 to 215° C. The pellets were mixed, added to the twin-screw extruder, and kneaded at a screw speed of 200 rpm.

A molten stranded product ejected from the extruder was cooled in water and formed into pellets by using a pelletizer. The pellets were dried in vacuum again at 60° C. and formed into an ISO dumbbell-shaped sample piece by using an injection molding machine (ROBOSHOT (trade name) α-100A type manufactured by FANAC) with a metal mold temperature set at 35° C. The test piece was placed in an oven at 90° C. for accelerating crystallization, and subjected to the heat distortion temperature (HDT) measurement, the Charpy impact test of notched sample, the tensile test, and the flexural test. The above crystallization at the temperature of 90° C. was carried out in accordance with the crystallizing method in which solidified moldings were heated to a temperature between the glass transition temperature and melting point. Table 1 collectively shows the results.

### Example 2

A test piece was prepared in the same manner as in Example 1, except that pellets containing 80 parts by weight of PLA-2 and 20 parts by weight of Copolymer-1 were used. Table 1 collectively shows the results of physical property tests of the test piece.

### Example 3

A test piece was prepared in the same manner as in Example 1, except that pellets containing 80 parts by weight of PLA-1 and 20 parts by weight of Copolymer-1 were used and the melt-kneading was conducted in a screw speed of 100 rpm. Table 1 collectively shows the results of the physical property tests of the test piece.

### Example 4

A test piece was prepared in the same manner as in Example 1, except that pellets containing 80 parts by weight of PLA-2 and 20 parts by weight of Copolymer-1 were used and the melt-kneading was conducted in a screw speed of 50 rpm. Table 1 collectively shows the results of the physical property tests of the test piece.

### Example 5

A test piece was prepared in the same manner as in Example 1, except that pellets containing 80 parts by weight of PLA-1 and 20 parts by weight of Copolymer-2 were used and the melt-kneading was conducted in a screw speed of 200 rpm. Table 1 collectively shows the results of the physical property tests of the test piece.

### Example 6

A test piece was prepared in the same manner as in Example 1, except that pellets containing 80 parts by weight of PLA-1 and 20 parts by weight of Copolymer-3 were used and the melt-kneading was conducted in a screw speed of 200 rpm. Table 1 collectively shows the results of the physical property tests of the test piece.

### Comparative Example 1

An ISO dumbbell-shaped sample piece was produced from 100 parts by weight of PLA-1 as raw material pellets by using an injection molding machine (ROBOSHOT (trade name) α-100A type manufactured by FANAC) with a metal mold temperature set at 35° C. The thus-obtained test piece was subjected to the heat distortion temperature (HDT) measurement, the Charpy impact test of notched sample, the tensile test, and the flexural test. Table 1 collectively shows the results of the physical property tests of the test piece.

### Comparative Example 2

A test piece was prepared in the same manner as in Comparative example 1, except that 100 parts by weight of PLA-2 were used as raw material pellets. Table 1 collectively shows the results of the physical property tests.

### Comparative Example 3

A test piece was prepared in the same manner as in Example 1, except that 100 parts by weight of PLA-1 were used as raw material pellets. Table 1 collectively shows the results of the physical property tests.

### Comparative Example 4

A test piece was prepared in the same manner as in Example 1, except that 100 parts by weight of PLA-2 were used as raw material pellets. Table 1 collectively shows the results of the physical property tests.

TABLE 1

| | | Remarks | | | | | | | | | |
|---|---|---|---|---|---|---|---|---|---|---|---|
| | | This invention | | | | | | Comparative example | | | |
| | | Sample No. | | | | | | | | | |
| | | | | | | | | Compar- | Compar- | Compar- | Compar- |
| | | | | | | | | ative | ative | ative | ative |
| | | Example 1 | Example 2 | Example 3 | Example 4 | Example 5 | Example 6 | example 1 | example 2 | example 3 | example 4 |
| (A) component | PLA-1 | 80 | — | 80 | — | 80 | 80 | 100 | — | 100 | — |
| (weight parts) | PLA-2 | — | 80 | — | 80 | — | — | — | 100 | — | 100 |
| (B) component | Copolymer-1 | 20 | 20 | 20 | 20 | — | — | — | — | — | — |
| (weight parts) | Copolymer-2 | — | — | — | — | 20 | — | — | — | — | — |
| | Copolymer-3 | — | — | — | — | — | 20 | — | — | — | — |

| | | | | | | | | | | |
|---|---|---|---|---|---|---|---|---|---|---|
| Screw speed (rpm) | 200 | 200 | 100 | 50 | 200 | 200 | — | — | 200 | 200 |
| Tensile modulus (GPa) | 1.6 | 1.6 | — | — | — | — | 1.9 | 1.9 | 2.2 | 2.4 |
| Flexural modulus (GPa) | 1.4 | 1.2 | — | — | — | — | 3.0 | 2.8 | 3.2 | 3.0 |
| Elongation at break (%) | 35 | 15 | — | — | — | — | 5 | 10 | 3 | 7 |
| HDT (° C.) | 60 | 60 | — | — | — | — | 55 | 56 | 62 | 62 |
| Charpy impact | 70 | 35 | 75 | 40 | 90 | 70 | 1.6 | 2.4 | 2.8 | 4.8 |
| value (kJ/m²) | | | | | | | | | | |

Comparisons between Example 1 and Comparative examples 1 and 3, and between Example 2 and Comparative examples 2 and 4 reveal that the poly(lactic acid)-containing compositions of the present invention, in which crystallization was accelerated, each exhibited a higher elongation at break (1.5 to 12 times) than the compositions formed solely of poly(lactic acid). The results indicate that brittle poly(lactic acid) was modified into a material capable of providing ductility. This point is one of important effects of the present invention.

Comparisons between Example 1 and Comparative example 1, and between Example 2 and Comparative example 2 reveal that the the poly(lactic acid)-containing compositions of the present invention, in which crystallization was accelerated, each had a heat distortion temperature (HDT) of about 5° C. higher than those, which was not subjected to crystallization acceleration, formed of poly(lactic acid). The results indicate that heat stability was improved.

Comparisons between Example 1 and Comparative example 3, and between Example 2 and Comparative example 4 reveal that the poly(lactic acid)-containing compositions of the present invention each had a heat distortion temperature (HDT) of about 2° C. lower than those of sole poly(lactic acid) subjected to heat treatment, thereby indicating that heat stability hardly changes even with the poly(lactic acid)-containing resin composition.

As the most important result of the present invention, comparisons between Examples 1, 3, 5 and 6 and Comparative examples 1 and 3, and between Examples 2 and 4 and Comparative examples 2 and 4 reveal that the compositions of the present invention each had a significantly improved impact value compared with those formed solely of poly(lactic acid). The impact values of the resin compositions of the present invention increased to about 7 to 60 times those of the compositions formed of amorphous poly(lactic acid) before heat treatment. These values are comparable to several times that of an ABS resin which is a standard resin derived from petroleum. In an impact test with a 4 J hammer by using a notched test piece, the test pieces of Comparative examples 1 to 4 were each completely broken, but the test pieces of Examples 1 to 6 were each not broken into two pieces. That is, the results suggest that brittle poly(lactic acid) can be used as a material hardly broken completely.

FIG. 1a shows an electron microscope photograph (TEM) of the resin composition used in Example 1 (resin composition produced from poly(lactic acid) (weight average molecular weight (Mw) of about 140,000 to 150,000) and Copolymer-1 (epoxy-modified polyethylene-series copolymer) in weight ratio of 80/20 at a screw speed of 200 rpm). FIG. 1b shows an electron microscope photograph of the resin composition used in Example 5 (resin composition produced from poly(lactic acid) (weight average molecular weight (Mw) of about 140,000 to 150,000) and Copolymer-2 (epoxy-modified polystyrene(S)-butadiene(B)-styrene(S)-series block copolymer, hydrogenation rate of 0%) in weight ratio of 80/20 at a screw speed of 200 rpm). FIG. 1c shows an electron microscope photograph of the resin composition used in Example 6 (resin composition produced from poly(lactic acid) (weight average molecular weight (Mw) of about 140,000 to 150,000) and Copolymer-3 (epoxy-modified polystyrene(S)-butadiene(B)-styrene(S)-series block copolymer, hydrogenation rate of about 50%) in weight ratio of 80/20 at a screw speed of 200 rpm). The photographs reveal that the dispersion state varied depending on the kind of component polymers used.

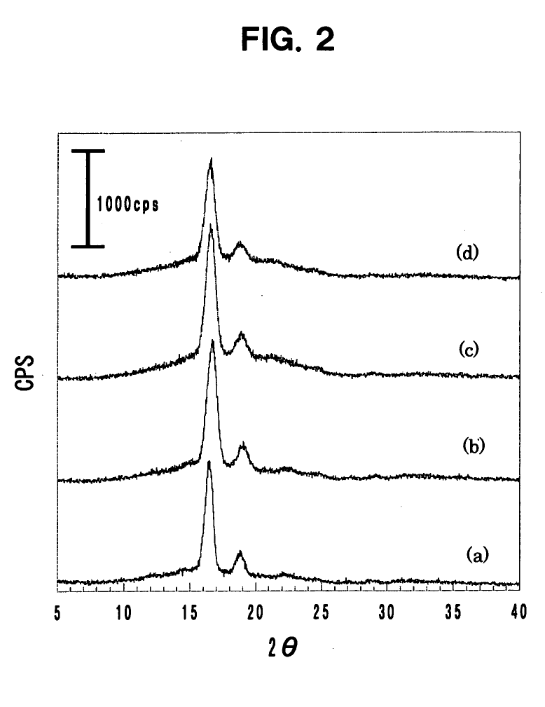

FIG. 2 shows wide angle X-ray diffraction (WAXD) profiles of: (a) the poly(lactic acid) (weight average molecular weight (Mw) of about 140,000 to 150,000); (b) the poly(lactic acid) (weight average molecular weight (Mw) of about 200,000); (c) the resin composition of the poly(lactic acid) (weight average molecular weight (Mw) of about 140,000 to 150,000)/epoxy-modified polyethylene-series copolymer in weight ratio of 80/20 produced at a screw speed of 200 rpm; and (d) the resin composition of the poly(lactic acid) (weight average molecular weight (Mw) of about 200,000)/reactive polyethylene-series copolymer in weight ratio of 80/20 produced at a screw speed of 200 rpm. The samples were each crystallized through heat treatment after injection molding. The results reveal that peaks of scattering from the (110) crystal plane and (203) plane of the α-type appeared at 2θ=16.5° and 19°, respectively.

Having described our invention as related to the present embodiments, it is our intention that the invention not be limited by any of the details of the description, unless otherwise specified, but rather be construed broadly within its spirit and scope as set out in the accompanying claims.

What we claim is:

1. A resin composition containing a crystal structure of poly(lactic acid), which is obtained by melt-kneading 30 to 99 mass % of poly(lactic acid) (A) and 70 to 1 mass % of a copolymer (B) having a functional group reactive with the poly(lactic acid) (A).

2. The resin composition according to claim 1, wherein the copolymer (B) is discontinuously dispersed as a dispersed phase with an average particle size of 10 μm to 10 nm in a continuous phase of the poly(lactic acid) (A).

3. The resin composition according to claim 1, wherein an X-ray diffraction profile of the resin composition designates a diffraction pattern with a peak that is characteristic of a crystalline substance of poly(lactic acid), when an X-ray diffraction strength of the resin composition is measured based on an angle (2θ) of a counter tube.

4. The resin composition according to claim 1, wherein a Debye ring is observed when the resin composition is irradiated with X-ray and the diffracted X-ray is received by a flat film.

5. The resin composition according to claim 1, wherein a heat of crystal fusion of the poly(lactic acid) (A) is 15 J/g or more in a DSC measurement at a scan rate of 10° C./minute.

6. The resin composition according to claim 1, wherein the poly(lactic acid) (A) has a weight average molecular weight of 5,000 to 1,000,000.

7. The resin composition according to claim 1, wherein the copolymer (B) is a copolymer having an epoxy group.

8. The resin composition according to claim 1, wherein the copolymer (B) comprises 0.1 to 30 mass % of an unsaturated glycidyl carboxylate unit and/or unsaturated glycidyl ether unit.

9. The resin composition according to claim 1, wherein the copolymer (B) has a structure of an olefin-based compound.

10. The resin composition according to claim 1, wherein the copolymer (B) is an epoxy group-containing ethylene copolymer comprising 60 to 99 mass % of an ethylene unit (a), 0.1 to 20 mass % of an unsaturated glycidyl carboxylate unit and/or unsaturated glycidyl ether unit (b), and 0 to 40 mass % of an ethylene-series unsaturated ester compound (c).

11. The resin composition according to claim 1, wherein a heat of fusion of the copolymer (B) is less than 3 J/g.

12. The resin composition according to claim 1, wherein a Mooney viscosity of the copolymer (B) is 3 to 70.

13. The resin composition according to claim 1, wherein the copolymer (B) is rubber.

14. The resin composition according to claim 13, wherein the rubber comprises a (meth)acrylate-ethylene-(unsaturated glycidyl carboxylate and/or unsaturated glycidyl ether) copolymer.

15. The resin composition according to claim 14, wherein the (meth)acrylate contains at least one selected from the group consisting of methyl acrylate, methyl methacrylate, n-butyl acrylate, n-butyl methacrylate, tert-butyl acrylate, tert-butyl methacrylate, 2-ethylhexyl acrylate, and 2-ethylhexyl methacrylate.

16. The resin composition according to claim 1, wherein the copolymer (B) is a block copolymer having a functional group reactive with poly(lactic acid) and comprising a block having at least one structure of a vinyl aromatic compound and a block having at least one structure of a conjugated diene compound.

17. The resin composition according to claim 1, wherein the copolymer (B) is a styrene-based elastomer.

18. The resin composition according to claim 1, wherein the copolymer (B) is a triblock copolymer having an epoxy group.

19. A method of producing a resin composition, comprising the steps of:
melt-kneading 30 to 99 mass % of poly(lactic acid) (A) and 70 to 1 mass % of a copolymer (B) having a functional group reactive with the poly(lactic acid); and
molding the kneaded composition under conditions for crystal growth.

20. The method of producing a resin composition according to claim 19, wherein a molding temperature is adjusted within a range of ±30° C. of a peak crystallization temperature of the resin composition, in which the peak crystallization temperature is obtained by reducing a temperature in a molten state of the resin composition through a differential scanning calorimetry (DSC).

21. The method of producing a resin composition according to claim 19, wherein the obtained resin composition is subjected to a heat treatment after molding, to accelerate crystallization.

22. Moldings, which are obtained by molding the resin composition according to claim 1.

23. The moldings according to claim 22, wherein the moldings are obtained by adjusting a molding temperature within a range of ±30° C. of a peak crystallization temperature of the resin composition, in which the peak crystallization temperature is obtained by reducing a temperature in a molten state of the resin composition through a differential scanning calorimetry (DSC).

24. The moldings according to claim 22, wherein the obtained moldings are subjected to a heat treatment after molding.

25. The moldings according to claim 22, wherein the molding is injection molding.

26. The moldings according to claim 22, wherein the molding is press molding.

27. The moldings according to claim 22, wherein the molding is extrusion molding.

28. The moldings according to claim 22, wherein the molding is film molding.

29. The moldings according to claim 22, wherein a shape of the moldings is any one of a film, a sheet, and a plate.

30. The moldings according to claim 22, wherein a shape of the moldings is any one of a net, a fiber, a non woven fabric, a woven fabric, and a filament.

31. The moldings according to claim 22, wherein a shape of the moldings is a rod or an irregular shape.

32. The moldings according to claim 22, wherein a shape of the moldings is any one of a tube, a pipe, a bottle, and a cylinder.

33. The moldings according to claim 22, wherein the moldings are used for any one of containers, home appliance components, transmission components, housing materials, toys, commodities, materials for agriculture or fishery, mobile device components, packaging materials, medical components, and automobile components.

34. Moldings, which are obtained by molding the resin composition produced by the method according to claim 19.

35. The moldings according to claim 34, wherein the moldings are obtained by adjusting a molding temperature within a range of ±30° C. of a peak crystallization temperature of the resin composition, in which the peak crystallization temperature is obtained by reducing a temperature in a molten state of the resin composition through a differential scanning calorimetry (DSC).

36. The moldings according to claim 34, wherein the obtained moldings are subjected to a heat treatment after molding.

37. The moldings according to claim 34, wherein the molding is injection molding.

38. The moldings according to claim 34, wherein the molding is press molding.

39. The moldings according to claim 34, wherein the molding is extrusion molding.

40. The moldings according to claim 34, wherein the molding is film molding.

41. The moldings according to claim 34, wherein a shape of the moldings is any one of a film, a sheet, and a plate.

42. The moldings according to claim 34, wherein a shape of the moldings is any one of a net, a fiber, a non woven fabric, a woven fabric, and a filament.

43. The moldings according to claim 34, wherein a shape of the moldings is a rod or an irregular shape.

44. The moldings according to claim 34, wherein a shape of the moldings is any one of a tube, a pipe, a bottle, and a cylinder.

45. The moldings according to claim 34, wherein the moldings are used for any one of containers, home appliance components, transmission components, housing materials, toys, commodities, materials for agriculture or fishery, mobile device components, packaging materials, medical components, and automobile components.
